(19) United States
(12) Patent Application Publication
(10) Pub. No.: US 2005/0282833 A1
(43) Pub. Date: Dec. 22, 2005

(54) Pharmacological treatment of psoriasis

(76) Inventor: Zehra Rowjee, Glenview, IL (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/523,782

(22) PCT Filed: Aug. 11, 2003
(86) PCT No.: PCT/US03/25142

§ 371 Date: Jul. 21, 2005

Related U.S. Application Data
(60) Provisional application No. 60/402,525, filed on Aug. 9, 2002.

(51) Int. Cl. A61K 31/522; A61K 31/513

(52) U.S. Cl. 514/263.35; 514/263.36; 514/269

(57) ABSTRACT

The present invention relates generally to the prevention of psoriatic-related disorders via administration of anti-viral agents or combination of anti-viral agents that possess anti-hyperproliferative pharmacological activity.

### STATEMENT OF PRIORITY

This application claims priority to U.S. Provisional Application 60/402,525, filed Aug. 9, 2002, which is incorporated by reference in its entirety.

### BACKGROUND OF THE INVENTION

1. Field of the Invention

This invention generally relates to methods for the pharmacological treatment of hyperproliferation of skin cells and, more specifically, to the administration of agents or compositions having an anti-proliferative keratinocyte effect for the alleviation of psoriasis (e.g., plaque and arthritic) and other psoriatic-related disorders.

2. Related Technology

Psoriasis is a benign disease of the human skin generally characterized by periodic flare-ups of sharply defined red patches covered by a silvery, flaky surface that affects an estimated 6.4 million Americans and affects about 3% of the world's population. The primary activity leading to psoriasis occurs in the epidermis wherein the pathological process starts where keratinocytes (immature skin cells) are produced within the basal (bottom layer of the epidermis) layer. Keratinocytes manufacture keratin, a tough protein that assists in formation of hair and nails as well as skin.

In normal cell growth, keratinocytes maturate and migrate from the bottom basal layer to the surface and are shed unobtrusively over a time period about a month. In comparison, psoriasis is characterized by rapid keratinocyte proliferation wherein the keratinocyte cells travel from the basal layer to the surface in only about four days. Psoriatic skin is unable to shed these rapidly migrating keratinocyte cells quickly enough so accumulation of these cells occurs and is characterized by thick, dry patches, or plaques. These plaques appear as silvery, flaky areas of dead skin on the epidermal layer (outer layer of the skin). In addition, the underlying layer, the dermis, is red and inflamed due to an increased blood supply to the abnormally multiplying keratinocytes.

The pathology of psoriasis is thought to originate from genetic abnormalities in the immune system that are triggered by environmental factors. Psoriasis occurs in many different varied forms of pathology. The three most common forms of psoriasis are plaque psoriasis, psoriatic arthritis, and guttate psoriasis.

Patches one-eighth inch in diameter, which gradually enlarge and thicken to develop silvery, flaky areas of dried plaques, characterize the onset of plaque psoriasis. If the plaque is scratched or scraped, bleeding spots the size of pinheads appear underneath known as an Auspitz sign. Some of the patches become ring shaped (annular) with a clear center and scaly raised borders that may be wavy or snake-like. Plaque psoriasis most often occurs on the elbows, knees, and the lower back. Plaques, however, can also appear on the palms, nails, genital areas of both women and men, thighs and calves of the legs, and soles of the feet. Plaque psoriasis rarely affects the face of adults, but about half of the patients suffer plaques in the scalp that may even extend down from the hairline to the forehead. In children, plaque psoriasis is most likely to start in the scalp and spread to other parts of the body including the face and the ears. Plaque psoriasis may persist for long periods, but more common are periodic flare-ups triggered by certain factors such as cold weather, infection, or stress.

Psoriatic arthritis is the second most common form of psoriasis and is defined as an inflammatory condition that is associated with psoriasis. It is not clear whether psoriatic arthritis is unique or is a genuine variation of psoriasis, but both appear to be autoimmune type responses. Psoriatic arthritis is characterized by stiff, tender, and inflamed joints and simultaneously occurs with skin flare-ups. Psoriatic arthritis usually is localized in the joints of the fingers and toes. About 80% of psoriatic arthritic patients have psoriasis in the nails. Psoriatic arthritis, however, can also occur in the knees, hip, elbows, and spine. When it affects the spine, psoriatic arthritis most frequently targets the sacrum (lower part of the spine). Although patients with psoriatic arthritis tend to have mild skin manifestations, the disease is systemic. Therefore, psoriatic arthritis is more serious than the common psoriatic condition and is estimated to affect 2% to as high as 42% of all psoriatic patients.

Guttate psoriasis is the third most common form of psoriasis and is defined as teardrop-shaped patches that erupt suddenly, usually over the trunk and often on the arms, legs, or scalp. Guttate psoriasis usually affects children and young adults, often about one to three weeks after a viral or bacterial (usually streptococcal) infection in the lungs or throat. A family history of psoriasis and stressful life events are also highly linked with the onset of guttate psoriasis. Interestingly, guttate psoriasis can also develop in patients who have had earlier forms of psoriasis. In such cases, it is more likely to emerge in people treated with widespread topical corticosteroid dressings.

Less common forms of psoriasis are, but not limited to, inverse psoriasis, seborrheic psoriasis, nail psoriasis, generalized erythrodermic psoriasis (psoriatic exfoliative erythroderm), and pustular psoriasis. All of these forms of psoriasis share a set of common factors wherein the immune system, enzymes, and other biochemical substances that regulate skin cells become impaired causing rapid keratinocyte proliferation and inflammation. Such abnormalities are likely due to one or more genetic defects often set off by environmental triggers. It is thought that a combination of one or more genes is involved with increasing a person's susceptibility to autoimmunity leading to psoriasis. For example, processes leading to most autoimmune diseases involve the human leukocyte antigen (HLA) system, which normally picks off parts of antigens and presents them on the surface of a cell so that factors in the immune system can recognize and destroy them. When the HLA system malfunctions, immune disorders such as psoriatic arthritis arise. Psoriatic disorders are highly associated with Europeans and North Americans who carry the HLA genetic factor, HLA-B27 (about 3% of Caucasian Americans). The rate of psoriatic disorders in African Americans is about 1% and is rarely seen in Native Americans. Researchers in the U.S., Canada, and Europe have now identified four key genes called PSORs 1-4 that are involved in psoriasis. Of particular interest are the genes located in regions on specific chromosomes that are linked to HLA and tumor necrosis factor, an immune component strongly associated with psoriasis.

Environmental or outside factors often trigger these genetic abnormalities leading to the onset and worsening of psoriatic pathology. Weather is a strong factor in psoriasis. Cold, dry weather is common precipitant of psoriasis flare-ups and occurs more frequently in African Americans, Japanese individuals, and Caucasians who live in colder climates than in people of any ethnicity who live in Africa. Stress, unexpressed anger, and emotional disorders, including despression and anxiety, are also strongly associated with psoriasis flares. Infections caused by viruses (such as HIV) or bacteria (often streptococcal infections of the upper respiratory tract) can trigger some cases of psoriasis. A Köbner response, or a delayed response at the site of injury, such as burns, cuts, injections, or other previous skin disorders are areas that psoriasis can develop. A number of drugs can worsen or induce pre-existing latent psoriasis including, but not limited to, anti-malarial drug chloroquine, drugs such as angiotensin-converting enzyme and beta blockers used to treat hypertension and heart problems, progesterone, lithium, and indomethacin (an anti-inflammatory drug). Withdrawal from oral steroids or high-potency steroid ointments that cover wide skin area can cause flare-ups of severe psoriasis, including guttate, pustular, and erythrodermic psoriasis. Interestingly, these topical ointments, which are used to treat psoriasis, can create a rebound and worsening pathology of psoriasis upon ending treatment.

A true animal model for psoriasis does not exist although rare primates with clinical and histopathological features of psoriasis have been reported (N. J. Lowe, Drug Dev. Res. 13:147-155 (1998)). Consequently, investigation of antipsoriasis drugs has relied on experimentally-induced hyperplasia in animals or mouse strain bearing the spontaneous mutation (fsn) for flaky skin (J. P. Sundberg et al., J. Invest. Dermatol. 92:414 (1989)). Another mouse model having epidermal proliferation is the essential fatty-acid deficient (EFAD) hairless mouse. Experimentally induced animal models also include athymic nude mice, which are immunologically defective, engrafted with diseases human skins.

Current therapies consist of efforts to reduce the rapid cell proliferation and to reduce inflammation. In general, the following three treatment options are used for psoriasis from least to greatest potency: (1) topical treatments including lotions, ointments, and shampoos are useful for mild-to-moderate psoriasis, but rarely complete clearance, however; (2) phototherapy including using ultraviolet B (UVB) or psoralen with ultraviolet A (PUVA), and more recently narrow band UVB; (3) systemic agents wherein this treatment employs various oral drugs that affect the whole body system, not just the skin.

The current treatments discussed generally above and in more detail below are only suppressive, not curative. When patients are provided with this information, along with the potential adverse effects that all of the current treatments offer, many patients exhibiting limited disease will often choose to live with their condition (Greaves et al., New England Journal of Medicine 332:581-588 (1995)). The goal in current treatments of patients suffering from psoriasis is to minimize the extent and severity of the disease to a point where it no longer disrupts the quality of the patients' life (i.e. local symptoms such as itching, pain, prominent lesions and psychological status such as embarrassment over appearance, fear of rejection). The current therapeutic options for psoriasis will be discussed in order from treating the most mild case of psoriasis to the most severe case of psoriasis. A treating physician's goal in treating psoriasis should be using the most effective, yet least damaging agent to the patient that is available.

Emollients

In mild cases of psoriasis, an emollient agent, such as mineral oil or paraffins in an oil-water emulsion, can be used. These agents hydrate the stratum corneum and facilitate desquamation. A combination of an emollient agent with moisturizing aqueous cream, such as betamethasone dipropionate cream can reduce the degree of dryness, scaling, and induration (Watsky et al., Cutis 50:383-386 (1992)). This combinational therapy is effective in inhibiting arachidonic acid oxidation by blocking the inflammatory cytokines associated with various psoriatic pathologies (Penneys et al., Brit. J. of Derm. 103:257-262 (1980)). Emollient agents are problematic because these agents are somewhat effective for only the most mild cases of psoriasis, and there is a lack of published comparative trials examining the efficacy of different emollients in psoriasis.

Salicyclic Acid

Salicyclic Acid is a keratolytic agent, which softens and dissolutes the horny layer of the epidermis. Combining salicyclic acid with coal tar, topical corticosteroids, or dithranol improve these agents efficacy to penetrate the skin, especially effective in areas such as the palms, soles, and scalp. Certain topical embodiments of the present invention may be combined with salicyclic acid to achieve maximum skin penetration and treatment of psoriatic skin. Salicyclic acid has not been demonstrated to act as a single agent for treatment of psoriasis and can act as an irritant when used in higher concentrations (60%>) (Greaves et al., at 588).

Coal Tar

Coal tar preparations can be applied to psoriatic areas of the body once or twice daily at an optimal concentration of 1 to 5%. When lesions are widespread, coal tar baths are useful. Coat tar can cause irritation so treatment regiments should begin at levels of 0.5 to 1.0% crude coal tar and increase concentrations upon favorable tolerance and efficacy of the patient. Coal tar is messy, stains clothing, and should be used with caution upon the face and flexures due to potential side effects of acneiform, eruptions, folluculitis, and photosensitivity. Coal tar shampoos for scalp psoriasis should also be used with caution due to the absorption of the polycyclic aromatic hydrocarbons present in coal tar, which are potentially carcinogenic (Kanzler et al., Brit. J. of Derm. 129:310-314 (1993)).

Diathranol

Dithranol (anthralin) is a chemical that is oxidized to form highly reactive free radical compounds that are thought to inhibit DNA synthesis. It is usually applied as a paste on the psoriatic lesions 24 hours after a daily coal tar bath and UVB phototherapy for maximum inhibition of keratinocyte DNA synthesis (Ryatt et al., Brit. J of Derm. 111:455-459 (1984)). Applying dithranol should only be long enough (15-60 minutes) for drug penetration of lesional, but not perilesional skin. Diathranol is an irritant and causes inflammation of the perilesional skin. In addition, diathranol stains the skin and clothing and should not be used in the face, flexures, or unstable psoriatic lesions.

Topical Corticosteroids

Topical corticosteroids are the most widely prescribed treatment for psoriasis in the United States. Corticosteroids in general have anti-inflammatory (vasoconstriction), anti-proliferative, and immunosuppressive activities, which are effective for countering psoriatic pathology. Unfortunately, many side effects from topical corticosteroids accompany patients during treatment regiments. The side effects include dermal atrophy, striae, telangiectasia, acneiform eruptions, perioral dermatitis, hypopigmentation, tachyphylaxis, masking of local infections, and possible systemic adsorption where upon adrenal gland is suppressed Katz et al., Dermatology Clinics 13:805-815 (1995)).

Vitamin D₃ Analogues

Vitamin D₃ analogues include calcipotriol and tacalcitol, which act predominantly by inhibiting epidermal cell proliferation and enhancing cell differentiation. Vitamin D₃ analogues are used to treat mild-to-moderate plaque psoriasis and are advantageous over older topical treatments (e.g., coal tar and dithranol) because they are more aesthetically acceptable to patients. Side effects common to vitamin D₃ analogues are lesional and perilesional irritation, which preclude use on facial lesions and the flexures (Ashcroft et al., J. of Clin. Pharm. and Therapeutics 25:1-10 (2000)). The invention disclosed herein, in contrast, indicates use for treatment of facial lesions and flexures.

Tazarotene

Tazarotene is a topical retinoid for treatment of mild-to-moderate plaque psoriasis affecting up to 10% of the skin surface. Tazarotene normalizes keratinocyte differentiation and proliferation by inducing expression of three tazarotene-induced genes in the epidermis (TIG) (Duvic et al., J. of the Am. Acad. of Derm., 37:S18-S24 (1997)). There are common adverse effects with this topical retinoid. These side effects include pruritus, burning, erythema, and irritation. Therefore, tazarotene should not be applied to the face, intertriginous areas, or the scalp. In addition, since retinoids are teratogenic, this drug cannot be used by patients who are pregnant or nursing.

For more serious conditions of psoriasis, the treatment options of UVB phototherapy, photochemotherapy (PUVA), methotrexate, cyclosporine, acitretin, and hydroxyurea have been used. Each type of systemic agent must be carefully monitored for potentially serious side-effects as will be discussed below.

Phototherapy

Phototherapy, specifically ultraviolet B radiation (UVB) is useful in the management of moderate to severe, guttate and chronic plaque psoriasis. A patient suffering widespread psoriatic lesions is exposed to UVB two to three times a week. After each treatment, the patient usually experiences minimal erythema. To minimize exposure to UVB radiation, cold tar, coconut oil, or some emollients (e.g., white and yellow paraffin) are used with phototherapy (Stern et al., J. of the Am. Acad. of Derm. 15:546-552 (1986)). Narrowband UVB phototherapy at the 311 nm region produces a greater improvement in psoriasis than broadband UVB sources (290-320 nm). Phototherapy in general must be carefully regulated, owing to the short-term risks of erythema and vesiculation and the long-term risks of premature skin aging. UVB treatment is not recommended for patients suffering from photosensitivity disorders such as lupus erythematosus.

Photochemotherapy

Photochemotherapy combines long wave (320-400 nm) ultraviolet A irradiation with the oral or topical administration of psoralens (PUVA). The two agents of psoralens are 5- and 8-methoxypsoralen (MOP), which intercalate into DNA. UVA radiation activates MOP and together this process disrupts DNA synthesis, thereby inhibiting cell proliferation. This method requires two precautions when treating patients. First, PUVA dose increments must be calculated to determine both optimal treatment regimens and the maximum phototoxic dose (MPD) that can be tolerated by the patient. Second, administration of PUVA causes a number of short term side effects and long term risks of disease. The short-term side effects include nausea, pruritus, erythema, acneiform eruptions, Koebner reaction, headache, and skin pain (Wolff, K., Brit. J. of Derm. 122:117-125 (1990)). Long term risks include the development of actinic keratoses, widespread PUVA lentigines, premature aging of the skin and irregular pigmentation. In addition, cataracts may occur in patients who fail to wear suitable UVA eye protection for 12-24 hours after oral psoralen ingestion. Long-term treatment increases risk of non-melanomatous skin cancer, particularly squamous cell carcinoma (Stern et al., Cancer 73:2759-2764 (1994)). There is also an increased risk of malignant melanoma (British Photodermatology Group, Brit. J. of Derm. 137:327-330 (1997)), and in men, there is a higher incidence of genital skin cancer (British Photodermatology Group, Brit. J. of Derm. 130:246-255 (1994)). Therefore, there is a need in the art for safer measures of treating psoriasis rather than the high-risk treatment of photochemotherapy.

Systemic Agents

Methotrexate (MTX) (brand name: Rheumatrex) is a folic acid antagonist that inhibits the enzyme dihydrofolate reductase, thereby blocking an essential step in DNA synthesis. MTX works well treating psoriasis by virtue of its immunodulatory properties. MTX is administered at a dosage level of 5-20 mg. Usually, a test dose of 2.5 to 5 mg is used to ensure the patient can tolerate the drug. If the test dose is tolerated, MTX is a preferred treatment for long-term use. Unfortunately, long-term treatment is accompanied by a risk of hepatotoxicity. Patients must avoid alcohol and if abnormal amino propeptide of type III procollagen (PIIINP) assay indicates an overactive liver, a liver biopsy may be required (Gawkrodger et al., J. of Derm. Treatment 8:27-55 (1997)). Other side effects include nausea, leucopenia, and thrombocytopenia. MTX has not been proven to be carcinogenic, but it is teratogenic and therefore, pregnant, breast-feeding patients, or patients planning on conceiving within 3 months should not use MTX (Boffa et al., Clin. Exper. Dermatology 21:399-408 (1996)). Drug toxicity can also result upon a combination of particular drugs with MTX. These particular drugs include salicyclates, nonsteroidal anti-inflammatory drugs (NSAIDs), penicillins, trimethoprim, cotrimoxazole, probenecid, acitretin, and cyclosporin and retinoids (Ashcroft et al., at 5).

Cyclosporin is used in treatment of severe psoriasis in patients wherein conventional therapy was ineffective. Cyclosporin inhibits the production of IL-2 and thereby T-cell activation by blocking cytoplasmic calcineurin phosphatase. Regular monitoring of the patient during treatment is recommended due to the concerns of toxicity and possible renal impairment. Other side effects include hypertension, hepatotoxicity, hypertrichosis, gingival hyperplasia, gastrointestinal disturbances, and central nervous system, including tremor and paraesthesia (Gawkrodger, D., J. of Derm. Treatment 8:27-55 (1997)). The concentration of cyclosporin in the blood must also be monitored, because many drugs affect the cyclosporin blood concentrations such as calcium-channel antagonists, doxycycline, erythromycin, oral contraceptive, ketoconazole, carbamazepine, rifampicin, phenyloin, phenobarbitone, amphotericin, ciprofloxacin, and grapefruit juice increase plasma cyclosporin concentrations, which could result in hepatotoxicity.

Acitretin is a synthetic retinoid for treatment of severe, recalcitrant psoriasis. Acitretin has been found to be most effective in treating erythrodermic or pustular psoriasis. This drug enhances the therapeutic effects of PUVA and UVB phototherapy while minimizing the dosage of each treatment. There are many side effects. Women of child-bearing years must use contraception for at least 2 years after withdrawal of therapy due to teratogenic properties of the drug. Other common adverse side effects include elevation of liver enzymes, skin peeling, and alopecia (Halioua et al., Brit. J of Derm. 122:135-150 (1990)). Hyperlipidaemia may occur at a higher risk in patients with a history of lipid disorders, high alcohol intake, diabetes, obesity, and smoking. Skeletal alterations associated with long term-use include ligamentous calcification and hyperostosis (Gollick et al., Brit. J. of Derm. 135:6-17 (1996)).

Hydroxyurea is generally considered a third-line alternative agent, used only in situations where other systemic agents have failed. The main concern is the risk of myelosuppression, which can manifest as megaloblastic anaemia, thrombocytopenia, or leucopenia (Gawkrodger et al., J. of Derm. Treatment 8:27-55 (1997)). Other side effects included diffuse hyperpigmentation, fever, alopecia, elevation of liver enzymes, and nausea. Patients need to be closely monitored during this drug regimen and the drug is best not used by women of childbearing age (Boyd et al., J. of Am. Acad. of Derm. 25:518-524 (1991)).

Systemic corticosteroids are the least effective systemic treatment. Risk for rebound flaring of psoriasis upon discontinuation may lead to severe, recalcitrant, generalized, pustular forms of the disease. Therefore, systemic corticosteroids are recommended only: (1) if persistent, uncontrollable erythrodermic psoriasis cause metabolic complications; (2) other drugs are ineffective in treating generalized pustalar forms; and (3) severe psoriatic polyarthritis threatens severe joint damage.

As discussed above, each one of the systemic treatment regimens is accompanied by a number of side effects and long-term risks. Even though some treatments are effective in treating recalcitrant psoriasis, the risk of hepatotoxicity and other side effects are common with all the current systemic treatments. Therefore, a novel treatment without toxicity is needed in the art.

Alternative Therapeutic Methods

Alternative, experimental means of treating psoriasis are presented below. There has been a focus using “biological compounds” to combat psoriasis. Various clinical studies have focused on pharmacological intervention by inhibition of co-stimulatory signals such as the CD80/CD86 pathway or activating epitopes such as CD2. Alternatively, the action of a soluble mediator can be blocked by masking its respective receptor (e.g., CD25/IL-2 receptor) or by inactivation of a mediator itself (e.g., tumor necrosis factor-α).

Monoclonal Antibodies and Fusion Proteins Used for Treating Psoriasis

Anti-CD4 antibodies have been investigated in placebo-controlled trials for purposes of treating patients suffering with severe psoriasis. Patients receiving the anti-CD4 humanized antibody OKTcdr4a reported tolerability to the antibody, but required a high-dose regimen in order to experience improvement of lesions after 3 months (Gottlieb et al., J. Am. Acad. Dermatol. 43:595-604 (2000)).

Another “biological compound” studied for future treatment of psoriasis has been the development of a chimeric anti-CTLA4-antibody (e.g., BMS-18867), which blocks the interaction between the co-stimulatory molecules CD80/CD86 and CD28-CD152, which disrupts the activation pathway of lymphocytes during antigen presenting cell-dependent T-cell stimulation. In a phase-1 trial, 9 out of 11 patients experienced a 50% improvement of psoriasis upon treatment with BMS-18867, which was sustained for up to 5 months (Abrams et al., J. Exp. Med. 192:681-694 (2000); Abrams et al., J. Am. Acad. Dermatol. 42:428-435 (2000)).

Targeting the interaction between ICAM1 of an antigen presenting cell and CD11a/LFA1 of a T-cell for purposes of treating psoriasis using an anti-CD11a-antibody hu1124) provided only moderate improvement in a high dose group during a 10 week trial. Treatment was also associated with headaches, chills, and fever in over half of the patients (Gottlieb et al., J. Am. Acad. Dermatol. 42:428-435 (2000)).

Another means of inhibiting T-cell dominated immune responses such as psoriasis has been masking the cellular receptor CD25 for IL-2. Two monoclonal antibodies, basiliximab and daclizumab, which have been used for therapy of transplant rejections, have been studied for their application to treating chronic plaque psoriasis. Results have been somewhat ineffective. Basiliximab was shown to provide mixed results wherein treatment of one patient with acute exacerbating psoriasis lead to long-lasting clearing of lesions for more than 10 weeks after last treatment, but a second patient with stable chronic plaque psoriasis did not show improvement of lesions with basiliximab treatment (Mrowietz et al., Arch. Dermatol. 136:675-676 (2000)). The second monoclonal antibody, daclizumab, was used in a clinical trial wherein only 30% of the patients experienced a reduction in severity of plaque psoriasis after 8 weeks of therapy (Krueger et al., J. Am. Acad. Dermatol. 43:448-458 (2000)).

TNF-α has been also been an interesting target for dampening the whole cascade of inflammatory responses in the skin. In one study, the monoclonal antibody infliximab had been designed to be directed against TNF-α directly. Infliximab proved to be highly effective in treating and sustaining clearing of pustular psoriasis. Because this antibody also has been registered to treat rheumatoid arthritis, investigators believe infliximab may also be effective in treating psoriatic arthritis (Mease et al., Lancet 356:385-390 (2000)). A second study used etanercept, a human recombinant solube p75 TNF-α receptor linked to the Fc portion of IgG1. Etanercept acts as a competitive inhibitor of TNF-α binding via cellular receptors. Patients using etanercept for 12 weeks experienced a mean improvement of psoriatic lesions of 46%. In addition, patients diagnosed with psoriatic arthritis experienced a 73% response to etancercept treatment (Mease et al. (2000)).

Other studies that have designed fusion proteins to reduce the number of T cells in the peripheral blood of psoriatic patients have demonstrated mixed results. One fusion protein called DAB₃₈₉IL-2 contained the receptor binding domain of IL-2 along with the membrane translocating and cytotoxic domains of the diphtheria toxin. This fusion protein was designed to eliminate activated T-cells characterized by high expression of IL-2 receptor (CD25). Clinical trials demonstrated psoriatic patients showed little improvement as compared with the placebo-treated group (Bagel et al., J. Am. Acad. Dermatol. 38:938-944 (1998)). A second example of fusion proteins designed for treatment of psoriasis is LFA3TIP. LFA3TIP is a fusion protein generated by combination of recombinant human LFA-3 with the Fc portion of human IgG1. Patients receiving LFA3TIP experienced plaque psoriasis improvements accompanied by a transient reduction in T-cell numbers in peripheral blood (Magilavey et al., J. Invest. Dermatol. 144:776 (2000).

These clinical studies demonstrate the inconsistent results experienced in the art when using antibodies or immunologically active fusion proteins. It is important to note, however, that investigators believe inactivation of a single pathway in the complex pathogenesis of psoriasis (e.g. use of basiliximab, anti-IL-2 receptor antibody) may not be enough in treating psoriasis. As demonstrated so far, there is still a need in the art for more “single-application treatments” that inactivate all the complex pathogenic pathways of psoriasis.

Cytokines and Mediators

Besides designing antibodies and fusion proteins to treat psoriasis, cytokines, mediators, and other compounds have been studied for application to psoriasis treatment through substance screening, drug-design, or experience in other diseases. For example, the cytokine IL-10 has been studied for its anti-psoriatic effects.

Increasing experimental evidence demonstrates that a psoriatic tissue reaction is at least in part a Th1-type response (Mrowietz, U., Clin. Derm. 26:362-367 (2001)). A recent clinical study has used IL-10, an inhibitor of Th1-type cell priming, but also a Th2-type mediator. Administration of IL-10 once daily over 24 days provided some improvement to psoriatic lesions of patients, but again demonstrates that IL-10 therapy may only be useful as a single-target treatment (Ellis et al., Arch. Dermatol. 114:776 (2000)).

Old Drugs, New Uses

Thiazolidinediones or glitazones are a group of substances that bind ligands for peroxisome proliferators-activated receptor-γ activation. Peroxisome proliferators-activated receptors activation is a member of the nuclear hormone receptor family (e.g., retinoic acid- and vitamin D-receptors). Thiazolidinediones and glitazones were developed to treat diabetes mellitus type 2, but have been shown to inhibit keratinocyte proliferation and down-regulate inflammatory mediators (Ellis et al., Arch. Dermatol. 136:609-616 (2000)). Other unique substances found effective for treating psoriasis are retinoids (e.g. bexarotene), which are used to treat cancer, and topical compounds called macrolactams (e.g., tacrolimus and ascomycins) used for treating an assortment of other dermatological aliments (Smit et al., J. Invest. Dermatol. 114:776 (2000); Rappersberger et al., J. Invest. Dermatol. 114:776 (2000)).

Employment of these agents for treatment of psoriasis can have significant side effects and economic costs depending upon the severity of the pathology of the particular psoriatic condition. Therefore, there remains a need for simple pharmacologically-based treatments that would offer benefits to a broad base of individuals suffering from psoriatic-related disorders. In addition, there is a need in the art to find an alternative viable treatment for psoriasis that would lend itself to a high rate of patient compliance, benefit, and healing of both emotional and physical wounds created by psoriatic-related disorders.

### SUMMARY OF THE INVENTION

The invention is directed to providing pharmacological treatment for the treatment or amelioration of psoriatic-related skin disorders.

In certain embodiments, a pharmaceutical composition comprising a therapeutically effective amount of a pharmaceutical comprising a nucleoside analog or prodrug thereof is administered to a patient diagnosed with a psoriatic-related skin disorder. Examples of psoriatic-related skin disorders include plaque psoriasis, psoriatic arthritis, guttate psoriasis, inverse psoriasis, seborrheic psoriasis, nail psoriasis, generalized erythrodermic psoriasis (psoriatic exfoliative erythroderm), and pustular psoriasis.

In some embodiments, the pharmaceutical composition comprises acyclovir or a prodrug thereof. In preferred embodiments, an acyclovir prodrug is administered orally at a dose of 1 gram to 3 grams per day.

In other embodiments, the invention relates to the use a nucleoside analog prodrug in a pharmaceutical composition for the treatment of a psoriatic-related disorder.

In some aspects, the invention relates to an article of manufacturing. The article of manufacturing comprises packaging material and a pharmaceutical composition contained within the packaging material. The pharmaceutical composition comprises an amount of nucleoside analog or prodrug thereof effective to treat a psoriatic-related skin disorder, and the packaging material comprises a label or package insert indicating that the pharmaceutical composition can be used for treating a psoriatic-related skin disorder.

### DETAILED DESCRIPTION OF THE INVENTION

The present invention is directed to methods for the treatment or amelioration of psoriatic-related skin disorders, the method comprising the administration of an therapeutically effective amount of an anti-viral agent to a patient in need of such therapy. Typically, the patient presents one or more symptoms associated with a psoriatic-related skin disorder such that the patient is diagnosed with such a disorder. Symptoms include, but are not limited to, psoriatic lesions and inflammation of one or more joints or the spine. A therapeutically effective amount can be an amount that alleviates the severity of one or more symptom or an amount that prevents the progression of one or more symptoms associated with psoriatic-related skin disorder. In preferred embodiments, a therapeutic effective amount is an amount sufficient such to cause a complete recovery from a psoriatic-related skin disorder, i.e., the patient presents no symptoms following treatment.

In certain embodiments, the anti-viral agent is a nucleoside analog (e.g., acyclovir) or a prodrug thereof (e.g., valacyclovir or analogs thereof). Effective antiviral agents typically have a restricted spectrum of antiviral activity and target a specific viral protein, most often an enzyme (polymerase or transcriptase) involved in viral nucleic acid synthesis. Other current agents (e.g. nucleoside analogs) inhibit active DNA replication via DNA chain termination. These antiviral drugs combat the replication strategy of either DNA viruses (such as poxviruses (small pox), herpesviruses (chickenpox, shingles, herpes), adenoviruses (conjunctivitis, sore throat), hepadnaviruses (hepatic B), and papillomaviruses (warts), RNA viruses (such as rubella virus (German measles), rhabdovinises (rabies), picornaviruses (poliomyelitis, meningitis, colds), arenaviruses (meningitis, Lassa fever), arboviruses (yellow fever, arthropod-borne encephalitis), orthomyxoviruses (influenza), and paramyxoviruses (measles, mumps), or retroviruses (such as HIV).

In certain embodiments, a nucleoside analog is administered to a patient. In preferred embodiments, a nucleoside analog prodrug is administered to a patient. Upon administration of the prodrug to the patient, it is converted to the active nucleotide analog. Acyclovir and valacyclovir are examples of a nucleotide analog and prodrug, respectively, which were designed to combat the replication cycle of herpes viruses.

Nucleoside analogs useful to combat herpes virus replication are well known in the art and their use within pharmaceutical compositions to treat a psoriatic-related skin disorder is specifically contemplated herein. Examples include gancyclovir, N-methanocarbathymidine (Noy et al., Cancer Chemother Pharmacol. Nov; 50(5):360-6. Epub 2002; Zalah et al, Antiviral Res. 2002 Jul; 55(1):63-75), Lobucavir (Smart and Torres, GMHC Treat Issues. 1996 August; 10(8):6-7), and acyclovir.

Acyclovir (9-[2-hydroxyethoxy)methyl]-9H-guanine) is an acyclic guanine nucleoside analog that lacks a 3′-hydroxyl on the side chain. Acyclovir has been shown to be most effective therapeutically against herpes simplex virus-1, approximately two-fold less active against herpes simplex virus-2,10-fold less potent against varicell-zoster virus (VZV) or Epstein-Barr virus (EBV), and least active against cytomegalovirus (CMV) or human herpesvirus (HHV-6) (Wagstaff et al., Drugs 47:153-205 (1994)). Acyclovir is available as capsules, as an ointment, and as a powder to be reconstituted for intravenous use.

There are several prodrug forms of acyclovir including valacyclovir and analogs thereof (Friedrichsen et al., Eur J Pharm Sci. 2002 July; 16(1-2):1-13). Valacyclovir is the L-valyl ester prodrug of acyclovir and is typically available for oral administration.

Acyclovir inhibits herpes DNA synthesis via a mechanism wherein acyclovir is converted to the monophosphate derivative by a herpesvirus thymidine kinase (product of early gene expression). For example, once acyclovir monophosphate (acyclovir-mp) is formed, acyclovir-mp is then recognized by cellular kinases, which phosphorylate acyclovir-mp to acyclovir-di-phosphate (acyclovir-dp) and, subsequently, to acyclovir-tri-phosphate (acyclovir-tp). Uninfected cells convert little or no acyclovir to the phosphorylated derivatives because the herpes virus thymidine kinase is not present. Thus, acyclovir is selectively activated in cells infected with herpesviruses that code for appropriate herpes thymidine kinases. In fact, the affinity of acyclovir for HSV thymidine kinase is about 200-fold greater than for the mammalian enzyme. In addition, cellular enzymes convert the monophosphate to acyclovir triphosphate, which is present in 40- to 100-fold higher concentrations in HSV-infected than in uninfected cells, and competes for endogenous deoxyguanosine triphosphate (dGTP). Incorporation of acyclovir-tp into the DNA primer strand during viral DNA replication leads to chain termination and formation of an inactive complex with the viral DNA polymerase. Acyclovir-tp acts essentially as a chain terminator because it lacks the 3′-hydroxyl group. This mechanism is termed suicide inactivation, the terminated DNA template containing acyclovir binds the enzyme and lead to irreversible inactivation of the DNA polymerase. Because valacyclovir is a prodrug of acyclovir, its mechanism of suicide inactivation is essentially the same as acyclovir.

Although not wanting to be limited by any particular mechanism of action, it is believed that the anti-viral agent, the present invention is directed to methods for the treatment or amelioration of psoriatic-related skin disorders comprising the administration of a dose of acyclovir or valacyclovir having an anti-hyperproliferative effect against rapidly dividing keratinocyte cells and prevents the inflammation therefrom.

A nucleoside analog or prodrug thereof may be administered alone to a patient in need or in a pharmaceutical compositions where it is mixed with suitable carriers or excipients at doses to treat or ameliorate a variety of disorders. Such a composition may also contain (in addition to protein and a carrier) diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier can depend on the route of administration.

Suitable routes of administration may, for example, include oral, topical, rectal, transmucosal, or intestinal administration, parenteral delivery, including intramuscular, subcutaneous, intramedullary injections, as well as intrathecal, direct intraventricular, intravenous, intraperitoneal, intranasal, or intraocular injections. Administration of a nucleoside analog or prodrug thereof (acyclovir or valacyclovir) can be carried out in a variety of conventional ways, such as oral ingestion, topical application or intravenous injection. Oral administration to the patient of valacyclovir or an analog thereof is preferred.

For oral administration, a nucleoside analog or prodrug thereof can be formulated readily by combining these active compounds with pharmaceutically acceptable carriers well known in the art. Such carriers enable the compounds of the invention to be formulated as tablets, pills, dragees, capsules, gels, liquids, syrups, slurries, suspensions, and the like, for oral ingestion by a patient to be treated. Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular fillers, such as sugars, including lactose, sucrose, mannitol, or sorbitol; cellulose preparations, such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintergrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coating for identification or to characterize different combinations of active compound doses.

Pharmaceutical preparations which can be used orally include push-fit capsules made of gelatin, as well as soft, sealed capsules made of gelatin and plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

All formulations for oral administration should be in dosages suitable for such administration. When formulated to release the agent over time (time-release), larger dosages may be administered as compared to formulations wherein the agent is quickly absorbed into blood system. In preferred embodiments, 500 mg of valacyclovir is administered orally. Administration may be repeated one or more times a day for several weeks. Daily dosages can be from 10 mg to 10 grams daily. In light of the present disclosure, those of skill in the art will be able to determine optimal dosages of the nucleoside analog or prodrug thereof without undue experimentation. In preferred embodiments, valacyclovir is administered orally at a daily dosage of at least 500 mg, at least 1 gram, at least 1.5 grams, at least 2.0 grams, at least 2.5 grams, at least 3.0 grams, at least 3.5 grams, at least 4.0 grams, or at least 5.0 grams.

For buccal administration, the compositions may take the form of tablets or lozenges formulated in conventional manner. Osmotic mini-pumps and timed-related pellets or other deposit forms of administration may also be used.

In certain embodiments, the antiviral agent (e.g., nucleoside analog) is administered in conjunction with one or more other psoriasis treatment (discussed in the Background of the Invention above). In preferred embodiments, side effects are limited by enabling administration of lower dosages than either treatment alone. The treatments may be co-administered or administered separately.

Embodiments of the invention include an article of manufacture. The article of manufacture contains a pharmaceutical composition comprising an antiviral agent, preferably a nucleoside analog or prodrug thereof. The article of manufacture can further contain a label or package insert indicating that the pharmaceutical composition can be used for treating a psoriatic-related skin disorder. The label or package may provide the indication by including an internet address, wherein the website corresponding to that address containing information regarding use of the pharmaceutical composition for treating a psoriatic-related skin disorder.

The following example illustrates the effects of administration of an antiviral agent, and in particular a nucleoside analog prodrug, to cause suppression of psoriatic symptoms.

Further aspects of the invention and embodiments will be apparent by those skilled in the art. In order that the present invention is fully understood, the following examples are provided by way of exemplification only and not by way of limitation.

Example 1 describes the agents or compositions that possess a specific psoriatic-related pharmacological activity that is used to effectively suppress or prevent psoriatic disorders and in particular, an anti-hyperproliferative pharmacological activity against rapid keratinocyte proliferating cells and the inflammation that results therefrom.

The following example is illustrative of aspects of the present invention, but is not to be construed to be limiting.

### EXAMPLE 1

### Suppression of Psoriasis-Case Study 1

A fifty-eight year old female patient with a history of plaque psoriasis over the last several years was diagnosed by her physician. Patient was treated with valacyclovir tablets at 500 mg taken 2 times a day for 20 days. Under the valacyclovir treatment, patient experienced complete recovery from the psoriasis. No reoccurrence of the psoriatic symptoms has appeared since treatment ended.

### EXAMPLE 2

### Suppression of Psoriasis-Case Study 2

A sixty-two year old male with a history of reoccurring plaque psoriasis over the last 30 years, as diagnosed by his physician, began treatment with Valtrex (valacyclovir) tablets at 500 mg taken 3 times a day for several weeks. Dosage of valtrex was increased to 1.0 gram taken 3 times a day for several weeks. Both the psoriatic lesions and other symptoms associated with psoriasis improved and cleared to 75-80% recovery.

### EXAMPLE 3

### Suppression or Prevention of Psoriasis

An individual diagnosed with a psoriatic-related disorder is administered either a composition or agent having any of the foregoing pharmacological profiles in an amount effective to prevent or suppress such disorders. The specific dose may be calculated according to such factors as body weight or body surface. Further refinement of the calculations necessary to determine the appropriate dosage for treatment of psoriatic-related disorders is routinely made by those of ordinary skill in the art without undue experimentation. Appropriate dosages may be ascertained through use of established assays for determining dosages. Routes of administration for the foregoing methods may be any systemic means including oral, intraperitoneal, subcutaneous, intravenous, intramuscular, transdermal, or by other routes administration. Osmotic mini-pumps and timed-released pellets or other depot forms of administration may also be used.

Finally, those of skill in the art will recognize that with respect to the compounds discussed above, such compounds may contain a center of chirality. Thus such agents may exist as different enantiomers of enantiomeric mixtures. Use of any one enantiomer alone or contained within an enantiomeric mixture with one or more stereoisomers is contemplated by the present invention.

Although the present invention has been described in terms of preferred embodiments, it is intended that the present invention encompass all modifications and variations that occur to those skill in the art upon consideration of the disclosure herein, and in particular those embodiments that are within the broadest proper interpretation of the claims and their requirements. All literature cited herein, where acceptable by the laws of a country or the policies of a patent office, is incorporated by reference.

1. A method for the treatment of a psoriatic-related skin disorder, the method comprising administering to a patient diagnosed with the disorder a therapeutically effective amount of a pharmaceutical composition comprising a nucleoside analog prodrug.

2. The method of claim 1, wherein the psoriatic-related skin disorder is selected from a group consisting of plaque psoriasis, psoriatic arthritis, guttate psoriasis, inverse psoriasis, seborrheic psoriasis, nail psoriasis, psoriatic exfoliative erthroderm, and pustular psoriasis.

3. The method of claim 1, wherein the pharmaceutical composition comprises an acyclovir prodrug.

4. The method of claim 3, wherein the pharmaceutical composition comprises valacyclovir or an analog thereof.

5. The method of claim 4, wherein the pharmaceutical composition is administered orally.

6. The method of claim 5, wherein the pharmaceutical composition is administered orally at a dosage between 1 gram and 3 grams per day.

7. A method for the treatment of a psoriatic-related skin disorder, the method comprising orally administering to a patient diagnosed with the disorder a therapeutically effective amount of a pharmaceutical composition comprising a nucleoside analog or a prodrug thereof.

8. The method of claim 7, wherein the pharmaceutical composition comprises acyclovir or an acyclovir prodrug.

9. The method of claim 8, wherein the pharmaceutical composition comprises valacyclovir or an analog thereof.

10. The method of claim 9, wherein the pharmaceutical composition is administered orally at a dosage between 1 gram and 3 grams per day.

11. The method of claim 7 wherein the psoriatic-related disorder is selected from the group consisting of plaque psoriasis, psoriatic arthritis, guttate psoriasis, inverse psoriasis, seborrheic psoriasis, nail psoriasis, generalized erythrodermic psoriasis (psoriatic exfoliative erythroderm), and pustular psoriasis.

12. Use of a nucleoside analog or a prodrug thereof in an oral pharmaceutical composition for the treatment of a psoriatic-related disorder.

13. The use of claim 12 wherein the nucleotide analog is acyclovir.

14. The use of claim 13 wherein the prodrug is valacyclovir or an analog thereof.

15. Use of a nucleoside analog prodrug in a pharmaceutical composition for the treatment of a psoriatic-related disorder.

16. The use of claim 15 wherein the nucleotide analog prodrug is a prodrug of acyclovir.

17. The use of claim 16 wherein the prodrug is valacyclovir or an analog thereof.

18. An article of manufacture comprising packaging material and a pharmaceutical composition contained within said packaging material, wherein the pharmaceutical composition comprises an amount of nucleoside analog prodrug effective to treat a psoriatic-related skin disorder, and wherein said packaging material comprises a label or package insert indicating that said pharmaceutical composition can be used for treating a psoriatic-related skin disorder.

19. The article of manufacture of claim 18, wherein the pharmaceutical composition comprises an amount of valacyclovir or analog thereof effective to treat a psoriatic-related skin disorder.

20. The article of manufacture of claim 18, wherein the label or package insert indicates that said pharmaceutical composition can be used for treating a psoriatic-related skin disorder selected from the group consisting of plaque psoriasis, psoriatic arthritis, guttate psoriasis, inverse psoriasis, seborrheic psoriasis, nail psoriasis, generalized erythrodermic psoriasis (psoriatic exfoliative erythroderm), and pustular psoriasis.
